(19) United States
(12) Patent Application Publication
(10) Pub. No.: US 2012/0288527 A1
(43) Pub. Date: Nov. 15, 2012

(54) ANTIALLERGIC AGENT

(75) Inventors: Mamoru Isemura, Shizuoka (JP); Akihiro Kaneko, Saitama (JP); Kensuke Yasui, Saitama (JP)

(73) Assignees: SHIZUOKA PREFECTURE PUBLIC UNIVERSITY CORPORATION, Shizuoka-shi, Shizuoka (JP); NISSHIN PHARMA INC., Chiyoda-ku, Tokyo (JP)

(21) Appl. No.: 13/519,541

(22) PCT Filed: Dec. 28, 2009
(86) PCT No.: PCT/JP2009/071769

§ 371 Date: Jun. 27, 2012

(51) Int. Cl. A61K 39/35; A61P 37/08

(52) U.S. Cl. 424/275.1

(57) ABSTRACT

An agent having antiallergic effects is provided, which is easily produced and ingested. A solvent extract of green soybean is used as an antiallergic agent. A solvent extract of green soybean having the effect of suppressing IgE production and the effect of shifting the Th1-Th2 balance to the Th1 side is useful as an antiallergic agent.

### TECHNICAL FIELD

The present invention relates to an antiallergic agent having the effect of suppressing IgE production and the effect of increasing the value of Th1/Th2, comprising a solvent extract of green soybean, and an antiallergic food or beverage containing the agent.

### BACKGROUND ART

Allergic disease has one of the highest rates of incidence of any disease in advanced countries. Particularly typical examples thereof include pollinosis, allergic conjunctivitis, bronchial asthma, and atopic dermatitis. Further examples thereof are rheumatism, inflammatory bowel disease, and food allergy. Among these diseases, the number of patients with pollinosis and atopic dermatitis has been rapidly increasing in recent years, and these diseases are becoming major social problems.

Allergic reactions are broadly classified into 4 types (types I to IV). Many allergic diseases are considered to occur due to type I allergy. Type I allergy is also referred to as immediate-type allergy. Allergic reactions due to type I allergy are said to occur due to the complex interaction of a plurality of factors: allergen-specific IgE antibodies excessively produced from B cells; chemical mediators such as histamine and leucotriene that are released from mast cells and basophils; a shift of the functional balance between Th1 cells and Th2 cells (hereinafter, referred to as “Th1-Th2 balance” in the Description), which are subtypes of helper T cells, to the Th2 side; and the like. Specifically, an allergen that has entered the body is incorporated into antigen-presenting cells and then degraded. Partial information thereof is presented via antigen presentation to T cells. T cells subjected to antigen presentation are differentiated into Th2 cells and activated, thereby producing cytokines such as IL-4 and thus activating B cells. IgE antibodies specific to the allergen are produced from the thus activated B cells. Then the IgE antibodies bind to Fc receptors existing on the surfaces of mast cells or basophils. If the allergen again enters the body to result in crosslinking of IgE antibodies on the surfaces of mast cells or basophils, chemical mediators such as histamines are produced and released in large amounts from within the cells, and leucotrienes and the like are further produced on the cell surfaces. The thus produced substances enhance blood vessel permeability, so as to induce edema or the excessive secretion of nasal discharge. The substances further cause smooth muscle construction, so as to induce airway contraction.

In contrast, it is now becoming clear that recent changes or the like in lifestyle or diet lead to a chronic shift of the Th1-Th2 balance to the Th2 side, contributing to a tendency of increase in allergic disease.

Examples of methods for improving allergic symptoms currently include: physical methods, such as methods for reducing the chances of contact with allergens such as pollen and house dust using masks, goggles, air cleaners, and the like and methods for specifying foods that act as allergens and preventing such foods from being ingested; and pharmacologic methods such as methods for suppressing allergic reactions using antiallergic agents such as an antihistamic agent that inhibits released histamines from binding to receptors, a steroid agent that suppresses inflammatory reactions, and a degranulation-suppressing agent that has the effect of stabilizing cell membranes or an agent for inhibiting leucotriene synthesis. However, it is extremely difficult to completely prevent contact with allergens. Moreover, antiallergic agents have side effects in many cases, and thus long-term daily use is limited.

On the other hand, attempts have been made to improve allergic symptoms through ingestion of foods exerting antiallergic effects instead of the use of these agents. Sweet tea, Japanese basil, and foods supplemented with β-glucan or the like are known as examples of such foods. However, these foods have limited effects of improving allergies and such effects are not sufficient, although they cause fewer side effects than pharmaceutical products. None of these foods have been able to exert sufficient effects.

Therefore, antiallergic foods that can be taken daily and are capable of effectively alleviating symptoms such as pollinosis are desired.

Soybean is known well as a raw material for tofu, soy sauce, fermented soybean (Natto), or the like. Attempts have been made to obtain functional ingredients from soybean.

For example, Patent Document 1 discloses a composition for preventing and/or treating allergic symptoms, comprising isoflavone and specific saponin as active ingredients. Patent Document 1 describes that these ingredients can be extracted from soybean. However, the content of isoflavone or saponin contained in soybean is low. Thus, it is necessary to extract isoflavone or saponin from hypocotyls, which is the part of the soybean in which the content thereof is thought to be particularly high, and then to concentrate it through a further complicated purification step. Furthermore, isoflavone and saponin are strongly bitter, and thus have a bad taste, so that they cannot be ingested continuously. Moreover, these ingredients can exert toxicity, so that they should be ingested while carefully controlling ingestion intervals and doses. Hence, they are problematic in that it is difficult to ingest them to obtain the effects.

Patent document 2 discloses an antiallergic composition comprising stachyose as an active ingredient that can be extracted from soybean. However, in soybean, content of the stachyose is low, and thus no effective ingredient can be obtained via a simple extraction process. Therefore, it is necessary to carry out a complicated purification step such as chromatography to concentrate stachyose to a concentration as high as 98%. Furthermore, stachyose exerts effects only on type IV allergy. Therefore, there is a problem that the effect of stachyose cannot be expected on immediate-type allergies (type I, type II, and type III allergies) such as IgE-related pollinosis, allergic conjunctivitis, bronchial asthma, and pruritus symptoms of atopic dermatitis, although its effects on delayed-type allergy (in which immune cells are involved) can be expected.

### Prior Art Documents

### Patent Documents

### Patent Document 1 JP Patent Publication (Kokai) No. 2007-197398 A

### Patent Document 2 JP Patent Publication (Kokai) No. 2003-321372 A

### SUMMARY OF THE INVENTION

### Problem to Be Solved by the Invention

An object of the present invention is to develop and provide natural materials that can be safely, inexpensively, and conveniently ingested on a daily and continuous basis, have a significant antiallergic effect, and are effective for preventing and/or improving various immediate-type allergic diseases, and particularly type I allergic diseases, such as pollinosis, allergic conjunctivitis, bronchial asthma, and atopic dermatitis.

### Means for Solving the Problem

As a result of intensive studies to achieve the above objects, the present inventors have found that an extract from green soybean or preferably a composition conveniently extracted from green soybean using water, alcohol, or hydrous alcohol exerts shows the extremely strong effects of decreasing the IgE level, compared with those extracted from other types of soybean such as yellow soybean. The present inventors have further found that the extract or the composition is useful for improving the shift of the Th1-Th2 balance to Th1 dominance (i.e., increasing the value of Th1/Th2) and preventing and/or improving various allergic diseases. The present inventors have also found that a highly effective antiallergic agent can be conveniently obtained using green soybean without complicated concentration, and thus have completed the present invention.

The present invention relates to the following (1) to (6).
(1) An antiallergic agent, comprising a solvent extract of a green soybean seed.
(2) The antiallergic agent according to (1), which suppresses IgE production.
(3) The antiallergic agent according to (1), which increases the value of Th1/Th2.
(4) The antiallergic agent according to any one of (1) to (3), wherein the solvent extract is a water extract.
(5) The antiallergic agent according to any one of (1) to (3), wherein the solvent extract is a hydrous ethanol extract.
(6) An antiallergic food or beverage, feedstuff, or cosmetic, comprising the antiallergic agent according to any one of (1) to (5).

### Effects of the Invention

According to the present invention, an antiallergic agent, which can be safely, inexpensively, and conveniently ingested on a daily and continuous basis, has a significant antiallergic effect, and is effective for preventing and/or improving various allergic diseases such as pollinosis, allergic conjunctivitis, bronchial asthma, and atopic dermatitis can be provided. The antiallergic agent of the present invention suppresses IgE production at the cellular level, so as to shift the Th1-Th2 balance to Th1 dominance. Hence, the antiallergic agent can exert its effects regardless of whether it is systemically administered via oral ingestion or locally administered. The antiallergic agent is extremely useful since unfavorable effects can be minimized and users can recognize the effects continuously.

### BEST MODES OF CARRYING OUT THE INVENTION

Preferred aspects of the present invention are as described in detail below.

### 1. Antiallergic Agent

An aspect of the present invention is an antiallergic agent. The antiallergic agent of the present invention is characterized in that it contains a solvent extract from green soybean seeds as an active ingredient.

### 1-1. Definition

The term “antiallergic” in the present invention refers to the prevention, treatment, reduction, or alleviation of various harmful effects resulting from Th2 dominance (a shift to the Th2 side) by shifting the Th1-Th2 balance of helper T cells from Th2 side to the Th1 side (Th1 dominance). Specifically, the term refers to prevention, treatment, reduction, or alleviation of smooth muscle contraction, enhanced blood vessel permeability, neutrophil migration, platelet aggregation, and the like induced by chemial mediators that are secreted from mast cells and the like as a result of the action of IgE antibodies, as well as various symptoms induced thereby, for example.

In addition, the above “Th1-Th2 balance” refers to the maintainance of a mechanism by which Th1 cells and Th2 cells mutually suppress and control differentiation from precursor cells (Th0 cells) to the other cells (Th1 or Th2). The expression “shift(ing) the Th1-Th2 balance to the Th1 side (Th1 dominance)” means to increase the value of Th1/Th2. Here, the term “the value of Th1/Th2” refers to the absolute or relative ratio of Th1 cells to Th2 cells. The absolute ratio can be calculated based on the numbers of Th1 cells and Th2 cells existing in a predetermined amount of peripheral blood. Also, the relative ratio can be calculated by measuring the expression level of or the amount of IFN-γ secreted from Th1 cells and the expression level of or the amount of IL-4 secreted from Th2 cells, and then comparing the results using a contrasting method.

Also, the above term “chemial mediator” refers to, for example, histamine or the like secreted from mast cells or basophils. Therefore, effects that are induced by IgE antibodies can be measured by, in addition to a method for directly measuring the blood IgE level, a method for measuring the levels of the above chemial mediators.

The term “green soybean” in the present invention is a generic name for various soybean (Glycine max) varieties that are characterized in that the seed coats and/or embryos of the mature seeds appear to be entirely or partially green. Examples of such a variety include Akitamidori, Kiyomidori, Echigomidori, Ohsodefuri, Otofukeohsode(furi), Ohsode-no-mai, Wasemidori, Suzukari, Aomaru-kun, Aome soybean, Akishimidori No. 1, Sousei, Aonyudou, Kokujin, Ayamidori, Shinanoaomame, Iwatemidori, Hiden, Hitashimame, Tenshin green soybean, and Seijin soybean (Chinese green soybean). In general, known green soybean varieties are: varieties characterized in that the seed coats and embryos appear to be green, such as Akitamidori; and varieties characterized in that only the seed coats appear to be green, such as Ohsodefuri. The green soybean of the present invention may be of any variety and is of preferably a variety characterized in that the seed coats and embryos appear to be green. The color of “hilum” is even varied among green soybean varieties, such as yellow, green, dark brown, and black, but is not particularly limited. The term “mature state” refers to a state at which seeds are sufficiently mature to have germinating capacity. In general, soybean seeds in an immature state referred to as “soybean in pods (eda-mame)” appear to be green regardless of yellow soybean or black soybean. Varieties that appear to be green only in such an immature state do not fall under the category of the green soybean of the present invention. The term “green” as used herein refers to color that is recognized by human eyes on the basis of reflected light with a wavelength of 490 nm-570 nm. Therefore, the concentration is not limited as long as the color is within the above wavelength range. Examples of the color include pale green, lime green, green, turquoise blue, dark green, and blackish green.

The above term “seeds” include seed coats and/or embryos and embryos include cotyledons and hypocotyls.

The term “solvent extract” in the present invention refers to a solution obtained by eluting an active ingredient having an antiallergic effect from green soybean seeds into a solvent, or a dried product (may be in the form of powders or solids) thereof. The solvent extract of the present invention preferably has a content of an active ingredient having an antiallergic effect per unit weight higher than the green soybean seeds by concentrating the active ingredient. However, an active ingredient having an antiallergic effect is not required to be highly purified and may be in a crude state such that other ingredients are mixed therein.

### 1-2. Method for Producing Solvent Extract of Green Soybean

A method for producing the solvent extract of green soybean to be used in the present invention is as described in detail below.

One, two, or more green soybean varieties may be used for solvent extraction. Furthermore, soybean (e.g., yellow soybean or black soybean) other than green soybean may be contained in a solvent extract as long as it does not deteriorate the effects of the present invention.

The mature state of green soybean to be used herein is not particularly limited. For example, immature soybean (namely, the state of soybean in pods (eda-mame)), mature soybean (including a dry state and an undried state), or a combination thereof can be used herein. Also, in addition to an entire seed, a seed coat alone, or an embryo alone may be used. Preferably, an entire seed is used. The shape of green soybean to be mixed with an extraction solvent is not particularly limited. Examples thereof include a state of intact seeds obtained from their pods, a fragmented or pulverized state of seeds, a squeezed state of seeds (obtained by extraction via compression), and combinations thereof.

A solvent to be used for extraction is preferably water or an organic solvent. Specifically, examples of water include: pure water, distilled water, tap water, acid water, alkaline water, and neutral water. Also, examples of an organic solvent include alcohol that is in the form of liquid at room temperature such as lower alcohols (e.g., methanol, ethanol, n-propanol, isopropanol, and n-butanol) and polyhydric alcohols (e.g., 1,3-butyleneglycol, propylene glycol, and glycerin); ethers such as diethyl ether and propylether; esters such as butyl acetate and ethyl acetate; ketones such as acetone and ethyl methylketone; hexane; and chloroform. These solvents may be used independently or in combinations of two or more types thereof. An example of such a combination is a hydrous organic solvent as described later. Among the above organic solvents, alcohol that is in the form of liquid at room temperature such as a lower alcohol having a carbon atom number of 1-4 is preferably used in view of operability and environment. Ethanol is preferably used in view of the safety of a residual solvent.

An organic solvent to be used in the present invention includes hydrous organic solvents containing aqueous ingredients. The content of an aqueous ingredient in the above hydrous organic solvent is generally 80% by volume or less, preferably 65% by volume or less, and is more preferably 50% by volume or less, in view of retaining the extraction efficiency at a high level. Such a hydrous organic solvent is preferably a hydrous alcohol that is an alcohol above further containing an aqueous ingredient, and is more preferably hydrous ethanol.

Extraction methods include, but are not particularly limited thereto, known methods such as a method that involves immersing green soybean in an extraction solvent and then stirring or refluxing the solution, and a supercritical-fluid extraction method.

Specific examples of an extraction method include a method that involves adding green soybean into a solvent at room temperature or a heated solvent under reduced pressure, normal pressure, or under increased pressure, and then carrying out extraction while immersing or stirring the solution, and a method that involves carrying out extraction while refluxing in a solvent. At this time, the temperature for extraction appropriately ranges from 5° C. to the same as or below the boiling point of the solvent. The time for extraction differs depending on the type of a solvent to be used (in the case of a hydrous organic solvent, further depending on the content of an aqueous ingredient) or extraction conditions. The time for extraction appropriately ranges from about 30 minutes to 72 hours. When extraction is carried out by refluxing, a solvent with a low boiling point is preferably used to prevent a green soybean extract from being denatured or thermally decomposed. Furthermore, extraction can be carried out by a supercritical-fluid extraction method using carbon dioxide and the like.

Subsequently, the mixture containing an extract and residues is subjected to filtration, centrifugation, or the like as necessary, so that an extract from which solid ingredients that are residues have been removed is obtained. In addition, removed solid ingredients may be subjected again to extraction, and this procedure may be repeated several times.

The thus obtained extract may be directly used as a solvent extract of green soybean or may be further subjected to a method such as concentration, freeze drying, or spray drying, as necessary, so that it may be used after drying or pulverization.

When an extract is dried, specifically any drying method may be employed, as long as it can be carried out under conditions where the solvent extract of green soybean is not denatured or thermally decomposed. For example, an excipient may be added if necessary. Examples of the method to be employed herein include filtration, centrifugation, centrifugal filtration, spray drying, spray cooling, drum drying, vacuum drying, and freeze drying. These methods may be employed independently or in combination.

### 1-3. Antiallergic Agent Comprising Solvent Extract of Green Soybean as Active Ingredient

The antiallergic agent of the present invention is characterized by containing as an active ingredient a solvent extract of green soybean obtained by the above production method. Specifically, a solvent extract of green soybean can be directly used independently or can be formulated into various forms such as pharmaceutical products, foods, or feedstuffs and thus used on a continuous basis as an antiallergic agent (ingestion and administration are included herein).

The dose of the antiallergic agent of the present invention generally prescribed ranges from 0.01 g to 100 g per day for an adult human on the basis of the dry mass of a solvent extract of green soybean obtained by the above production method. In the case of peroral administration, the general daily dose ranges from 0.1 g to 50 g. The dose can be further increased since the extract is derived from soybean and thus has high safety. The daily dose can be administered once or in several separated instances of administration.

Regarding the antiallergic agent of the present invention, a solvent extract of green soybean can not only be directly used independently as a medicine, a food, a feedstuff, or a cosmetic, but also be used as a pharmaceutical composition in combination with, for example, an additive described later, another known immunostimulating substance and/or immunomodulating substance, or the like, as long as it does not inhibit the effects of the present invention. Examples of a formulation to be used as a pharmaceutical composition include: oral preparations such as tablets, capsules, granules, powders, syrups, dry syrups, solutions, and suspensions; enteral preparations such as inhalers and suppositories; skin external preparations such as paste, cream pharmaceuticals, gel, and adhesive preparations; drops; and injection preparations. Oral preparations are particularly used.

Such a formulation can be produced according to a conventional method by mixing a solvent extract of green soybean, that is an active ingredient, with a commonly used additive such as an excipient, a disintegrator, a binder, a lubricant, a surfactant, an alcohol, water, a water soluble polymer, a sweetener, a flavoring agent, and an acidulant depending on the formulation. In addition, a liquid preparation such as a solution or a suspension may be in a form that can be dissolved or suspended in water or another appropriate medium immediately before administration. Furthermore, when a formulation is in the form of tablets or granules, the surfaces thereof may be coated with sugar or the like by a known method.

The content of a solvent extract of green soybean in a pharmaceutical composition differs depending on the formulation. In the case of an oral preparation, the content generally ranges from 0.001% to 99% by mass, and preferably ranges from 0.01% to 80% by mass on the basis of the dry mass. The content is desirably determined so that the daily dose can be controlled to comply with the above daily dose per adult human.

### 1-4. Effect

The effect of the present aspect is as follows. The antiallergic agent of the present invention is directly used independently or used on a continuous basis as a pharmaceutical product, and the increase in INF-y and the decrease in IL-4 can shift the Th1-Th2 balance of helper T cells to the Th1 side. Specifically, the value of Th1/Th2 increases and the IgE level decreases, so that the antiallergic agent is useful for: preventing and/or improving various damages or diseases (e.g., various symptoms resulting from type I allergy such as pollinosis and atopic disease; autoimmune diseases, immunosuppression due to tumor, lowered immunity due to treatment with an anticancer agent or radiotherapy, and lowered immunity associated with acquired immune deficiency syndrome (AIDS), infectious diseases due to various bacteria, aging or pathological conditions), health enhancement, and acceleration of nutritional fortification.

Furthermore the antiallergic agent of the present invention is produced using green soybean to be used as a food as a raw material, and thus it is highly safe and has good flavor. Thus, the antiallergic agent can be directly and independently ingested orally and can be administered on a daily continuous basis for a long time period. Furthermore, the antiallergic agent can also be easily ingested on a continuous basis for a long time period in various forms of pharmaceutical products, foods, or feedstuffs. Moreover, the active ingredient of the antiallergic agent of the present invention can be conveniently extracted from green soybean via solvent extraction alone and is highly effective, so that it is also economically advantageous.

In the present invention, the Th1-Th2 balance of helper T cells is shifted to the Th1 side. Hence, the present invention is useful for: improving an immunosuppressive state resulting from tumor growth; improving a state of lowered immunity resulting from treatment with an anticancer agent or radiotherapy; preventing or improving acquired immune deficiency syndrome (AIDS); preventing or improving infectious diseases resulting from various types of bacteria; preventing or improving various symptoms derived from type I allergy; improving various symptoms and defects such as improvement of lower immunity associated with aging or pathological conditions; health enhancement; and accelerating nutritional fortification.

### 2. Antiallergic Food or Beverage, Feedstuff, and Cosmetic

Other aspects of the present invention are antiallergic foods or beverages, feedstuffs, and cosmetics. The antiallergic food or beverage, feedstuff, and cosmetic of the present invention are characterized by containing the antiallergic agent of the above aspect. Each composition is as described below.

### 2-1. Food or Beverage and Feedstuff

The antiallergic food or beverage and feedstuff of the present invention (hereinafter, referred to as “the antiallergic food or beverage and the like of the present invention”) are a food or beverage and feedstuff containing as an active ingredient the antiallergic agent of the present invention. When the antiallergic agent is prepared as a food or beverage or the like, the form thereof is not particularly limited. Examples thereof include all foods or beverages or feedstuffs with which the antiallergic agent of the present invention can be mixed, in addition to health foods, functional foods, foods for specified health use, or feedstuffs, pet foods, and the like for domestic animals, race horses, ornamental animals, and the like. Specifically, examples of various dosage forms of the food or beverage include tablets, chewable tablets, dust formulations, capsules, granules, drinkable preparations, and fluid diets such as tube feeding/enteral feeding products. The food or beverage in such a dosage form can be produced by a method similar to that for the above pharmaceutical composition. The food or beverage may be further prepared as: a beverage such as a tea beverage (e.g., green tea, oolong tea, or black tea), a soft drink, a jelly drink, a sports drink, a milk drink, a carbonated drink, a fruit juice beverage, a lactobacillus beverage, a fermented milk beverage, a powder beverage, a cocoa drink, or purified water; or spreads such as butter, jam, furikake (seasoned dried food for sprinkling on rice), and margarine, mayonnaise, shortening, custard cream, dressings, bakery goods, boiled rice, noodles, pasta, miso soup, tofu, milk, yogurt, soups or sauces, or sweets (e.g., biscuits or cookies, chocolate, candies, cake, ice cream, chewing gum, and tablet), for example.

The feedstuff of the present invention can also be used with a composition and/or in a form almost similar to that of the above food or beverage of the present invention. Hence, the description concerning the food or beverage in the Description can be similarly applied for the feedstuff.

Foods can be further produced according to a conventional method by mixing other food materials to be used for food production, various nutrients, various vitamins, minerals, amino acids, various fats and oils, various additives (e.g., taste components, sweeteners, acidulants such as organic acid, surfactants, pH regulators, stabilizers, antioxidants, pigments, flavor), and the like. Foods according to the present invention can also be produced by mixing a generally consumed food with the agent of the present invention.

The content of a solvent extract of green soybean in food differs depending on the formulation. The content generally ranges from 0.001% to 80% by mass, preferably ranges from 0.01% to 50% by mass, and more preferably ranges from 1% to 50% by mass on the basis of the dry mass. The daily ingestion dose may be ingested at once or in several separated instances of ingestion. The daily ingestion dose is desirably determined so that the daily ingestion dose per adult human can be controlled and taken.

### 2-2. Cosmetics

The antiallergic agent of the present invention can also be used as a cosmetic. When a cosmetic is prepared, a solvent extract of green soybean may be directly used as a cosmetic, or it may be prepared in a form such as: an emulsion, a cosmetic liquid, a cream, a lotion, an essence, a pack or sheet, a foundation, a face powder, a blush, lip rouge, eye shadow, eyeliner, or mascara; cleansing preparations such as a facial cleansing agent, a skin cleansing preparation, a gel, a gel agent, an agent for beautiful skin, or a body shampoo; a hair cosmetic such as a shampoo and a rinse; a preparation for the hair; a hair treatment; a baldness remedy; a bath agent; a paste; a quasi-drug; or face oil blotting paper, wherein a solvent extract of green soybean has been mixed via a general method.

Cosmetics can be produced according to a conventional method by appropriately mixing a conventionally known excipient or aroma chemical, fats and oils, a surfactant, an antiseptic, a sequestrant agent, a water soluble polymer, a thickener, a powder constituent such as a pigment, a UV protective agent, a moisturizing agent, an antioxidant, a pH regulator, a washing agent, a drying agent, an emulsifier, and the like, depending on a desired formulation in addition to a solvent extract of green soybean.

The content of a solvent extract of green soybean in a cosmetic is not particularly limited, generally ranging from 0.001% to 80% by mass and preferably ranging from 0.01% to 50% by mass on the basis of dry mass.

A food or beverage, a feedstuff, or a cosmetic containing the antiallergic agent of the present invention may be prepared by mixing the agent with, in addition to the above examples, conjugated linoleic acid, taurine, glutathione, carnitine, creatine, coenzyme Q, glucuronic acid, glucuronolactone, a capsicum extract, a ginger extract, a cacao extract, a guarana extract, a garcinia extract, teanin, γ-aminobutyric acid, capsaicin, capsiate, various organic acids, flavonoids, polyphenols, catechins, a xanthine derivative, nondigestible oligosaccharide such as fructooligosaccharide, or polyvinylpyrrolidone, for example.

The amount of such an additive to be mixed therewith may be appropriately determined depending on the form of the composition, the type of the additive, and a desired ingestion dose, ranging from 0.01% to 70% by mass and preferably ranging from 0.1% to 50% by mass in the agent, food, feedstuff, or cosmetic of the present invention.

### 2-3. Effect

The food or beverage, feedstuff, or cosmetic of the present invention contains the antiallergic agent of the present invention in the above aspect. If it is used on a continuous basis, the effects similar to those exerted by the above antiallergic agent can be obtained. Specifically, the value of Th1-Th2 increases and the IgE level decreases, so that various disorders and/or diseases such as pollinosis, atopic disease, and autoimmune disease can be prevented and/or improved. Also, the agent of the present invention is produced using green soybean to be used as a food, as a raw material, so that it has high safety and good flavor. The agent can be directly ingested orally or used as a cosmetic. Thus, the agent can be easily ingested on a continuous basis for a long time period in the form of a food or a feedstuff.

### EXAMPLES

The present invention will be described in more detail below with reference to examples. However, the examples are only given for illustrative purposes and the present invention is not limited thereto.

### Example 1

### Production of Water Extract

100 mL of Water was added to 10 g of crushed green soybean and then the mixture was heated at 90° C. for 60 minutes for extraction. Insoluble matter was removed by filtration and then water was evaporated, so that 2.2 g of powder was obtained.

### Example 2

### Production of Hydrous Ethanol Extract

100 mL of Hydrous ethanol (ethanol:water=95:5) was added to 10 g of crushed green soybean and then the mixture was shaken at 100 rpm at room temperature for 24 hours for extraction. Insoluble matter was removed by filtration and then the solvent was evaporated, so that 1.2 g of powder was obtained.

### Example 3

### Production of Water Extract

12 L of Water was added to 1 kg of crushed green soybean (Echigomidori) and then the mixture was heated at 95° C. for 60 minutes for extraction. Centrifugation was carried out to remove the precipitate, the supernatant was filtered to remove insoluble matter, the resultant was freeze-dried, and thus 346 g of powder was obtained.

### Comparative Example 1

In a manner similar to that in Example 2, extraction was carried out from soybean (yellow soybean), black soybean, red pea, red kidney, and chickpea using hydrous ethanol, so that powder was obtained from each type of soybean.

### Comparative Example 2

### Production of Water Extract of Yellow Soybean

Four liter of Water was added to 400 g of crushed yellow soybean (Fukuyutaka) and then heated at 95° C. for 60 minutes for extraction. Centrifugation was carried out to remove the precipitate, the supernatant was filtrated to remove insoluble matter, the resultant was freeze-dried, and thus 145 g of powder was obtained.

### Test Example 1

### Effect of Decreasing IgE Level (1)

100 μL of an antigen solution consisting of a suspension of 1 μg of ovalbumin (hereinafter, referred to as OVA) and 2 mg of an adjuvant (aluminium hydroxide) was intraperitoneally administered as 1^{st} sensitization to each mouse (Balb/c, ♂). One week later, 100 μL of the above suspension was intraperitoneally administered again as 2^{nd} sensitization. After the 2^{nd} sensitization, blood was collected from a mouse tail and OVA-specific IgE level in the blood was measured by an enzyme antibody method using a measurement kit (DS mouse IgE ELISA (OVA); Dainippon Sumitomo Pharma Co., Ltd.). Mice were divided into two groups as a control group and a green soybean group to be administered green soybean without bias of the IgE levels. Mice were bred with the following test feedstuffs. The control group was fed with AIN-76 feedstuff (Table 1) and the green soybean group was fed with AIN-76 feedstuff wherein the content of the hydrous ethanol extract of green soybean in Example 2 was 5%. The initiation date for the administration of the test feedstuff was designated as day 0. On days 0, 7, and 16, blood was collected from a mouse tail, and then the OVA-specific IgE level in the blood was measured. Table 2 shows the results.

TABLE 1
Composition of AIN-76 purified feedstuff

| | |
|---|---|
| Sucrose | 50% |
| Corn starch | 15% |
| Casein | 20% |
| Corn oil | 5% |
| AIN-76 vitamin mix | 1% |
| AIN-76 mineral mix | 3.5% |
| DL-methionine | 0.3% |
| Cellulose | 5% |
| Choline bitartrate | 0.2% |
| Total | 100% |

TABLE 2
OVA-specific IgE level in blood (μg/mL)

| | Day 0 | Day 7 | Day 16 |
|---|---|---|---|
| Control | 0.041 | 0.183 | 1.081 |
| Green soybean | 0.042 | 0.141 | 0.905 |

As is clear from Table 2, increments of IgE levels in blood of mice sensitized with OVA decreased due to the antiallergic agent of the present invention.

### Test Example 2

### Effect of Decreasing IgE Level (2)

200 μL of antigen solution consisting of a suspension of 1 μg of OVA and 2 mg of an adjuvant (aluminium hydroxide) was intraperitoneally administered as 1^{st} sensitization to each mouse (Balb/c, ♂, 6-week-old). Two weeks later, 200 μL of the above suspension was intraperitoneally administered again as 2^{nd} sensitization. One week after the 2^{nd} sensitization, blood was collected from a mouse tail and then OVA-specific IgE level in the blood was measured by an enzyme antibody method using a measurement kit (DS mouse IgE ELISA (OVA); Dainippon Sumitomo Pharma Co., Ltd.). Mice were divided into 3 groups as a control group, a green soybean water extract (from Example 3) group to be administered the water extract of green soybean, and a yellow soybean water extract (from Comparative example 2) group to be administered the water extract of yellow soybean without bias of the serum IgE levels. The grouping date was designated as day 0. Mice were bred with the following test feedstuffs. On and after day 2, the control group was fed with AIN-76 feedstuff shown in Table 1, and the test groups were fed with feedstuffs supplemented with the extract in Example 3 and the extract in Comparative example 2, respectively, so that the content of the extract was 5% in each feedstuff. The blood was collected on day 14 in mid-course and mice were dissected on day 28. The OVA-specific IgE level in the blood of each mouse was measured. Also, total IgG levels on day 28 were measured to observe the effects on antibodies of other classes. Table 3 shows the results.

TABLE 3
OVA-specific IgE level and total IgG level in blood (μg/mL)

| | | Day 0 | Day 14 | Day 28 |
|---|---|---|---|---|
| IgE | Control | 0.27 | 1.48 | 1.51 |
| | Green soybean | 0.27 | 0.52 | 0.34 |
| | (Example 3) | | | |
| | Yellow | 0.30 | 1.27 | 0.98 |
| | soybean | | | |
| | (Comparative | | | |
| | example 2) | | | |
| IgG | Control | — | — | 137.7 |
| | Green soybean | — | — | 158.9 |
| | (Example 3) | | | |
| | Yellow | — | — | 159.6 |
| | soybean | | | |
| | (Comparative | | | |
| | example 2) | | | |

As is clear from Table 3, the antiallergic agent (green soybean extract) of the present invention significantly suppressed increments of IgE levels in blood of mice sensitized with OVA, and the effect was extremely better than that of yellow soybean. Also, no significant changes were observed in IgG levels compared with the control group. It was demonstrated that the antiallergic agent of the present invention does not act specifically on the IgE antibody to induce a side effect.

### Test Example 3

### Effect of Decreasing IgE Level (3)

200 μL of an antigen solution consisting of suspension of 1 μg of OVA and 2 mg of an adjuvant (aluminium hydroxide) was intraperitoneally administered as 1^{st} sensitization to each mouse (Balb/c, ♂, 6-week-old). One week later, 200 μL of the above suspension was intraperitoneally administered again as 2^{nd} sensitization. One week after the 2^{nd} sensitization, blood was collected from a mouse tail. OVA-specific IgE level in the blood was measured by an enzyme antibody method using a measurement kit (DS mouse IgE ELISA (OVA); Dainippon Sumitomo Pharma Co., Ltd.). Mice were divided into 3 groups as a control group, a green soybean hydrous ethanol extract (Example 2) group to be administered the green soybean hydrous ethanol extract, a green soybean water extract (Example 1) group to be administered the green soybean water extract without bias of the serum IgE levels. Mice were bred with the following test feedstuffs. The control group was fed with AIN-76 feedstuff shown in Table 1 and the test groups were fed with feedstuffs supplemented with the extract of Example 1 or 2, so that the content of the extract was 5% in each feedstuff. The initiation date for the administration of the feedstuff was designated as day 0. Mice were dissected on day 24 and blood OVA-specific IgE levels were measured. Table 4 shows the results.

TABLE 4
OVA-specific IgE level in blood (μg/mL)

| | Example 2 | Example 1 |
|---|---|---|
| | Hydrous ethanol | Water extract |
| Control group | extract group | group |
| 1.77 ± 0.38 | 1.53 ± 0.51 | 1.02 ± 0.32 |

As is clear from Table 4, increments of IgE levels in blood of mice sensitized with OVA decreased due to the antiallergic agent of the present invention and particularly the water extract exerted high effects.

### Test Example 4

### Effect of Increasing IFN-γ Gene Expression

A cell line (Jurkat cells) was cultured in DMEM (GIBCO) supplemented with 10% FBS under conditions of 37° C. and 5% CO₂. Cells were diluted with a culture solution to 3×10⁵ cells/mL, the diluted solution was dispensed to a 24-well plate at 1 mL per well. 100 μL of each hydrous ethanol extract from the various pulses of Example 2 and comparative example was added to a culture solution at a final concentration of 500 μg/mL, followed by 48 hours of culture. After completion of culture, intracellular IFN-γ gene expression was measured using a quantitative PCR measuring device (Thermal cycler Dice TP-800; Takara Bio Inc). Table 5 shows the results represented by relative values obtained when the result for the control to which no solvent extract had been added was determined to be 1.

TABLE 5
IFN-γ gene expression level (relative value)

| | Example 2 | Comparative example | | | | |
|---|---|---|---|---|---|---|
| | Green | | Black | | Red | |
| Control | soybean | Soybean | soybean | Red pea | kidney | Chickpea |

| | | | | | | |
|---|---|---|---|---|---|---|
| 1.00 | 4.63 | 1.06 | 1.12 | 1.28 | 1.31 | 1.08 |

It was understood from Table 5 that soybean types other then green soybean had exerted almost no effect on IFN-γ. On the other hand, the antiallergic agent of the present invention significantly increased the expression level of IFN-γ, which is the indicator for Th1, and thus had the effect of shifting the Th1-Th2 balance to the Th1 side.

### Test Example 5

### Effect of Suppressing IL-4 Gene Expression

In a manner similar to that in Test example 4, a cell line (Jurkat cells) was cultured. A culture solution was dispensed to a 24-well plate, and then 100 μL of the hydrous ethanol extract from green soybean of Example 2 was added to a final concentration of 250 μg/mL or 500 μg/mL, followed by 48 hours of culture. After completion of culture, intracellular IL-4 gene expression was measured using a quantitative PCR measuring device (Thermal cycler Dice TP-800; Takara Bio Inc). Table 6 shows the results represented by relative values obtained when the result for the control to which no solvent extract had been added was determined to be 1.

TABLE 6
IL-4 gene expression level (relative value)

Example 2

| Control | 250 μg/mL | 500 μg/mL |
|---|---|---|
| 1.00 | 0.70 | 0.45 |

It was understood from Table 6 that the antiallergic agent of the present invention significantly decreased the expression level of IL-4, which is the indicator for Th2, and thus had the effect of shifting the Th1-Th2 balance to the Th1 side.

### Test Example 6

### Effect of Suppressing IgE Level and IL-4 Level

100 μL of an antigen solution consisting of suspension of 1 μg of OVA and 2 mg of an adjuvant (aluminium hydroxide) was intraperitoneally administered as 1st sensitization to each mouse (Balb/c, ♂). On day 14, 100 μL of the above suspension was intraperitoneally administered again as 2^{nd} sensitization. After the 2^{nd} sensitization, blood was collected from a mouse tail and then the OVA-specific IgE levels in the blood were measured by an enzyme antibody method using a measurement kit (DS mouse IgE ELISA (OVA); Dainippon Sumitomo Pharma Co., Ltd.). Mice were divided into 3 groups as a control group, a green soybean group to be administered green soybean, and a soybean group to be administered soybean without bias of the IgE levels. Mice were bred with the following test feedstuffs. The control group was fed with AIN-76 feedstuff, the green soybean group was fed with the AIN-76 feedstuff in which the content of the hydrous ethanol extract from green soybean of Example 2 was 5%, and the soybean group was fed with AIN-76 feedstuff in which the content of the hydrous ethanol extract from soybean of comparative example 1 was 5%. The initiation date for the administration of the test feedstuffs was designated as day 0. After 3 weeks, the IL-4 gene expression levels in Peyer's patch were measured using a quantitative PCR measuring device (Thermal cycler Dice TP-800; Takara Bio Inc). Table 7 shows the results represented by relative values obtained when the result for the control to which no solvent extract had been added was determined to be 1.

Furthermore, spleen cells were collected and then cultured for 72 hours. OVA-specific IgE levels in culture supernatants were measured by an enzyme antibody method using a measurement kit (DS mouse IgE ELISA (OVA); Dainippon Sumitomo Pharma Co., Ltd.). Table 8 shows the results.

TABLE 7
IL-4 gene expression level of Peyer’s patch (relative value)

| | Example 2 | Comparative |
|---|---|---|
| | Green | example |
| Control | soybean | Soybean |
| 1.00 | 0.71 | 0.86 |

TABLE 8
IgE level in spleen cells (μg/mL)

| | Example 2 | Comparative |
|---|---|---|
| | Green | example |
| Control | soybean | Soybean |
| 6.20 | 3.93 | 5.23 |

It was understood from Table 7 that the antiallergic agent of the present invention significantly decreased the expression level of IL-4, which is the indicator for Th2, compared with soybean types other than green soybean, and thus had the effect of shifting the Th1-Th2 balance to the Th1 side. Similarly, it was understood from Table 8 that the antiallergic agent of the present invention decreased increments of IgE levels in spleen cells sensitized with OVA, compared with soybean types other than green soybean.

### Example 4

### Production of Tablets

84 g of the water extract of green soybean obtained in a manner similar to that in Example 1, 10 g of crystalline cellulose (Asahi Kasei Corporation), and 5 g of polyvinylpyrrolidone (BASF) were mixed. 30 mL of Ethanol was added to the mixture, and then granules were produced by a wet method according to conventional techniques. Granules were dried and then 1.2 g of magnesium stearate was added to prepare granular powders for tablet making. Tablet making was carried out using a tableting machine and thus 100 tablets (1 g per tablet) were produced.

### Example 5

### Production of Granules

100 g of a hydrous ethanol extract of green soybean obtained in a manner similar to that in Example 2, 100 g of lactose (DMV), and 40 g of crystalline cellulose (Asahi Kasei Corporation) were mixed. 130 mL of ethanol was added to a kneading machine and then the resultant was kneaded in the kneading machine by a general method for 5 minutes. After completion of kneading, the resultant was sieved (10 mesh) and then dried in a dryer at 50° C. After drying, granulating was carried out, so that 240 g of granules was obtained.

### Example 6

### Production of Syrup

750 g of saccharose and 100 g of a water extract of green soybean obtained in a manner similar to that in Example 1 were added to and dissolved in 400 g of the boiled purified water while stirring the solution. The resultant was strained through a cloth in a hot state. Purified water was added to the resultant to a total amount of 1000 mL, so that a syrup was produced.

### Example 7

### Production of Fluid Diet

40 g of casein sodium (DMV), 160 g of maltodextrin (Sanwa starch), and 50 g of a hydrous ethanol extract of green soybean obtained in a manner similar to that in Example 2 were added to and dissolved in 700 g of pure water at about 65° C. Vitamin mix and a mixture of micromineral ingredients were added. The thus obtained mixture was put in a homomixer, and then rough emulsification was carried out at about 8,000 rpm for 15 minutes. The thus obtained emulsified solution was cooled to about 20° C., an aroma chemical was added, and then the solution was diluted with pure water to a final volume. A pouch was filled with 230 g of the solution, the pouch was sealed while performing nitrogen substitution. Sterilization was carried out at 121° C. for 15 minutes, and thus a fluid diet was obtained.

### Example 8

### Production of Bread

160 g of wheat flour (hard flour) was mixed with 2 g of dry yeast. Separately, 5 g of a water extract of green soybean obtained in a manner similar to that in Example 1, 25 g of table sugar, 3 g of common salt, and 6 g of powdered skim milk were dissolved in 70 g of warm water, one hen egg was added, and then the mixture was mixed well. The mixture was added to the above mixture of wheat flour and dry yeast. After kneading well by hand, about 40 g of butter was added and then the mixture was further kneaded well, so that the dough for 20 pieces of roll bread was prepared. Subsequently, the dough for these bread pieces was fermented, beaten egg was applied to the surface, and then it was baked in an oven at 180° C. for about 15 minutes, so that roll bread was prepared.

### Example 9

### Production of Confectionary

Margarine and table sugar were mixed, the mixture was stirred well using a mixer, and then whip was prepared. A half of a whole egg was added to the whip to prepare whipped cream. The water extract of green soybean obtained in Example 3 was added to and gently mixed with the whipped cream, thereby preparing dough. The dough was shaped and then baked in an oven at 150° C. for 25 minutes, so that a confectionary was prepared.

### Example 10

### Production of Confectionary

Whole eggs were beaten with an egg beater, and a sweetener in an amount corresponding to 90 g of table sugar was added to and mixed well with the resultant. 10 g of the water extract of green soybean obtained in Example 3, 40 g of wheat flour, and baking powder were added to the resultant. After stirring and mixing, butter and rum were further added to and mixed well with the solution, thereby preparing dough. The dough was placed in molds and then baked in an oven at 170° C. for 15 minutes, so that a confectionary was prepared.

### Example 11

### Production of Retort Rice

Rice cooking was carried out by adding 2 g of the water extract of green soybean obtained in Example 3 to about 360 mL of rice and water in a volume generally appropriate for such amount of rice. A retort pack was filled with cooked rice according to a commonly used method, and the pack was sealed while performing nitrogen substitution. Sterilization was carried out at 121° C. for 15 minutes, so that retort rice was obtained.

### Example 12

### Production of Pasta Sauce

150 g of a pasta meat sauce for one person was added to a pan, 1 g of a hydrous ethanol extract of green soybean obtained in a manner similar to that in Example 2 was put in the pan, and then the mixture was heated and mixed. A pouch was filled with the sauce and then sealed while performing nitrogen substitution. Sterilization was carried out at 121° C. for 15 minutes, so that a pasta meat sauce was obtained.

### Example 13

### Production of Vegetable Juice

A hydrous ethanol extract of green soybean obtained in a manner similar to that in Example 2 was added to and mixed with commercial vegetable juice to 5% by mass, so that vegetable juice was prepared.

### Example 14

### Production of Consomme Soup

100 g of onion, 100 g of carrot, 100 g of green onion, 50 g of celery, and 100 g of tomato were put in a pan. 500 g of ground beef, egg white (two eggs), and 1 kg of beef bouillon were added to the pan, and the resultant was then heated. When the resultant came to a boil, the flame was lowered. The resultant was then cooked on low heat for 1 hour while the scum and fat were removed from the surface. 50 g of a water extract of green soybean obtained in a manner similar to that in Example 1 was added and then the resultant was further cooked for 30 minutes, following which the solution was strained through a cloth, so that consomme soup was obtained.

### Example 15

### Production of Paste (% by Mass)

### Solution A

Hydrous ethanol extract of green soybean obtained in Example 2: 1
Propylene glycol: 5
Methyl parahydroxybenzoate: 0.2
Carboxyvinyl polymer: 0.5

### Solution B

Diisopropyl adipate: 10
Cetanol: 2
Polyoxyethylene hydrogenated castor oil 60: 2
Polyethylene glycol monostearate: 1
Butyl parahydroxybenzoate: 0.1

### Solution C

Purified water: appropriate volume (up to 100% by mass)

The solution B was mixed while heating and dissolving it at 70° C., so as to obtain an oil phase. The solution A was mixed while heating and dissolving it at 70° C., the oil phase of the solution B was added to and mixed with the solution A for emulsification. Thereafter, the resultant was cooled while adding the solution C and the mixture was mixed well, so that paste was obtained.

1. An antiallergic agent, comprising a solvent extract of a green soybean seed.

2. The antiallergic agent according to claim 1, which suppresses IgE production.

3. The antiallergic agent according to claim 1, which increases the value of Th1/Th2.

4. The antiallergic agent according to claim 1, wherein the solvent extract is a water extract.

5. The antiallergic agent according to claim 1, wherein the solvent extract is a hydrous ethanol extract.

6. An antiallergic food or beverage, feedstuff, or cosmetic, comprising the antiallergic agent according to claim 1.
